(19) United States
(12) Patent Application Publication
(10) Pub. No.: US 2026/0165652 A1
(43) Pub. Date: Jun. 18, 2026

(54) Multifunctional Garment with Therapeutic, Recreational, and Digital Interaction Features

(71) Applicant: Kristi Ann Langslet, Cranston, RI (US)

(72) Inventor: Kristi Ann Langslet, Cranston, RI (US)

(21) Appl. No.: 19/321,361

(22) Filed: Sep. 8, 2025

Related U.S. Application Data
(60) Provisional application No. 63/734,269, filed on Dec. 16, 2024.

(51) Int. Cl. A61B 5/00; A61B 5/11

(52) U.S. Cl. CPC A61B 5/6804; A61B 5/0022; A61B 5/1126

(57) ABSTRACT

A multifunctional elastic garment is disclosed that integrates compressive features, imaginative play elements, and optional digital interaction. The garment includes a body portion and at least one pair of closed-end pockets configured to receive the wearer's hands and forearms. Each pocket includes a flip-able edge to selectively cover or uncover the hands. Application of force within the pockets generates compressive pressure across the head, arms, and torso through stretchable fabric, without the use of straps or rigid elements. In certain embodiments, the garment may include graphic embellishments, detachable accessories, and sensor modules that are embedded or removably attached to monitor biometric, motion, or location data and wirelessly interface with external devices.

### CROSS-REFERENCE TO RELATED APPLICATIONS

This application claims the benefit of U.S. Provisional Patent Application No. 63/734,269 filed Dec. 16, 2024, the entirety of which is incorporated herein by reference.

### STATEMENT REGARDING FEDERALLY SPONSORED RESEARCH OR DEVELOPMENT

Not applicable.

### REFERENCE TO SEQUENCE LISTING, A TABLE, OR A COMPUTER PROGRAM LISTING APPENDIX

Not applicable.

### BACKGROUND OF THE INVENTION

### Field of the Invention

The present invention pertains to the field of apparel, specifically a multifunctional garment that integrates therapeutic sensory input, imaginative costume play, and an interactive user interface for digital applications. This garment features a pair of closed-end pockets strategically positioned to receive the wearer's hands and forearms. Each pocket includes a flip-able pocket edge, allowing the wearer to uncover or cover their hands at will, offering additional flexibility and control over the sensory experience. The garment combines self-directed proprioceptive compression for therapeutic benefits with customizable accessories for creative expression, while also offering the potential for integration with wearable technology for gaming and health monitoring purposes.

### Background

Many garments provide either functional or decorative benefits, but few integrate multiple roles in a single wearable item. Existing therapeutic compression garments often lack adjustability and aesthetic appeal, while themed costumes do not typically offer sensory or therapeutic advantages. There is a need for a garment that seamlessly combines adjustable therapeutic compression, customizable appearance, and optional integration with electronic devices for interactive experiences.

### SUMMARY OF THE INVENTION

The present invention relates to a multifunctional garment that combines therapeutic benefits, imaginative play, and digital interactivity. The garment is designed from a stretchable, elastic fabric (e.g., spandex-blend knit) that provides self-adjustable compression, offering proprioceptive input and deep pressure touch to the wearer's body, including the head, arms, and torso. This compression promotes body awareness, enhances focus, and supports emotional regulation, with the wearer able to control the intensity and duration of the pressure through force applied to the garment's strategically positioned closed-end pockets.

Each pocket is equipped with a flip-able pocket edge, allowing the wearer to uncover or cover their hands, thereby providing additional control over the garment's sensory effects. The wearer can adjust the compression based on their needs, offering a personalized therapeutic experience without relying on external devices like straps or inflatable elements.

The garment further supports creative expression by being customizable with various graphic embellishments, including printed images, embroidered patches, or sewn-on fabric additions. These designs allow for a broad range of themes and characters, facilitating imaginative play and role-playing scenarios. The garment may also be equipped with detachable accessories such as animal ears, wings, tails, or crowns, which can be attached or removed using mechanical fasteners, allowing the wearer to transform the garment into a unique costume. Additionally, the garment can include interactive sensor modules capable of monitoring biometric, location, and/or motion data. These sensors can wirelessly transmit data to external devices, enabling integration with digital applications for real-time feedback, interactive gaming, or fitness tracking.

With its combination of therapeutic features, customizable designs, detachable accessories, and potential for digital interaction, this invention offers a versatile garment that enhances sensory experiences, supports creative play, and engages with digital applications, making it suitable for various therapeutic, recreational, and interactive uses.

### ADVANTAGES OF THE INVENTION INCLUDE

Self-directed therapeutic compression without straps, buckles, or rigid elements.
Modular design supporting imaginative play and thematic customization.
Optional digital integration for health monitoring, gaming, and interactive applications.
Durable and comfortable construction for long-term use across multiple settings.

### INDUSTRIAL APPLICABILITY/UTILITY

The invention has applicability in the fields of therapeutic apparel, wearable sensory support, recreational costumes, and interactive digital garments. It may be used for therapeutic regulation in clinical, educational, or home environments, for entertainment and costume play, and for health monitoring or gaming when integrated with sensor-based technologies.

### BRIEF DESCRIPTION OF THE DRAWINGS

FIG. 1: A front perspective view of one embodiment of the multifunctional garment, showing the hood, closed-end pockets, and hands covered inside the pockets

FIG. 1A: A front perspective view of the garment of FIG. 1, showing the flippable pocket edges inverted to allow hands to be uncovered

FIG. 2: Front view of the garment with hands and arms applying force to the closed pockets, arms are crossed in front of the wearer for increased proprioceptive and tactile sensory experience

FIG. 3: Front view of the garment hands covered in the enclosed pockets, showing the placement of sensor modules, which may attach to the inside of the garment. Indicators show where detachable accessories can be applied

FIG. 4: Rear view of the garment with hands covered in the enclosed pockets, exerting force outwards for increased proprioceptive and tactile sensory experience FIG. 4A: Rear view of the garment with hands uncovered so hands can be used freely.

Indicators show where detachable accessories can be applied

FIG. 5: Rear view of the garment with hands covered in the enclosed pockets, with attached ears and tail accessories, facilitating imaginative play

FIG. 6: Pattern of garment prior to assembly

### REFERENCE TO FIGURES

The accompanying drawings, which are incorporated in and constitute a part of this specification, illustrate exemplary embodiments of the present invention and, together with the detailed description, serve to explain the principles of the invention. In the drawings, like reference numerals refer to like elements throughout the several views. The drawings are not necessarily to scale, and certain features may be exaggerated or simplified for clarity of illustration.

### DETAILED DESCRIPTION OF THE INVENTION

The present invention relates to multifunctional garments configured to provide therapeutic sensory input, support imaginative play, and optionally serve as an interactive interface for digital applications. While the embodiments described herein focus on garments constructed from stretchable textiles, the invention is not limited to any particular material, method of construction, particular sewing pattern, or method of configuration except as defined in the appended claims.

### 1. General Structure

In one embodiment, the multifunctional garment (10) includes a body portion covering at least the torso of the wearer, an optional hood (12) covering the wearer's head, and one or more strategically positioned pockets (20). The garment may be unitarily formed or assembled from multiple fabric panels joined by stitching, thermal bonding, adhesive bonding, or other suitable joining techniques.

The garment may be constructed from a resilient, elastic fabric such as a spandex-blend knit, elastane-containing jersey, warp-knit elastic textile, or any other stretchable material capable of repeated extension and recovery without significant loss of elasticity.

### 2. Therapeutic Compression Features

In one embodiment, each pocket (20) is closed at its distal end to receive the wearer's hands and forearms (30). When the wearer exerts force against the interior of the pockets and/or an integrated forearm cradle (30), the fabric stretches to generate compressive pressure across the head, shoulders, arms, and torso. The level of compression can be varied by the wearer through changes in the amount and duration of applied force, allowing self-directed sensory input without the need for external straps, buckles, inflatable chambers, or rigid frames.

Each pocket (20) may include a flippable pocket edge (18), which can be inverted to move between a closed position covering the hands and an open position exposing them. Alternative configurations include fold-over flaps, zippered openings, removable pocket covers, or elasticized slits. This adjustability enables the wearer to control both hand accessibility and the degree of compression.

In one embodiment, the forearm cradle (30) is formed by folding a lower edge of a fabric panel (32) toward the hood (12) and attaching it along a diagonal seam (24) extending from a first attachment point (38) to a second attachment point (40). The portion between point (40) and the cradle remains open, defining a sleeve edge (22) through which the wearer's hands may extend.

### 3. Customization and Imaginative Play Features

The garment (10) may be configured with interchangeable or fixed character designs (28), thematic embellishments, or decorative motifs. These can be applied through printing, embroidery, applique, heat transfer, woven patterning, or sewn-on fabric additions. In various embodiments, the garment may transform into costumes or character representations such as animals, mythical creatures, superheroes, or other themed designs.

Accessories (28), such as ears, tails, crowns, wings, fins, capes, or themed appendages, may be removably attached via hook-and-loop fasteners, snaps, buttons, zippers, magnets, or slotted channels. Such features support imaginative role-play while preserving the garment's therapeutic functionality.

### 4. Sensor Integration and Digital Interaction

In some embodiments, the garment (10) includes embedded or detachable sensor modules (26) capable of detecting biometric parameters (e.g., heart rate, skin temperature, galvanic skin response), motion (e.g., posture, activity level, orientation), or location data. These sensors may be integrated into the garment lining, mounted in removable housings, or positioned within accessory elements.

The sensor modules (26) may transmit data wirelessly (e.g., via Bluetooth®, Wi-Fi, NFC) or through wired connections to an external device such as a smartphone, tablet, or computer. The garment may thereby interface with software applications providing real-time feedback, interactive gameplay, therapeutic tracking, or health monitoring.

### 5. Materials and Durability

Suitable materials for the garment include but are not limited to: elastane-containing textiles, performance athletic fabrics, warp-knit stretch fabrics, or multi-layer laminates incorporating breathable membranes. In one embodiment, the garment material is selected to maintain elasticity and tensile strength after repeated cycles of stretching and recovery.

The garment may be produced in various sizes, proportions, and configurations for children, adults, athletes, or individuals with sensory processing needs. It may be machine washable, hand washable, or constructed from components that can be separated for cleaning.

### 6. Alternative Embodiments

Without limiting the scope of the invention, alternative embodiments may include:
Garments without a hood, with a partial hood, or with detachable head coverings.
Pockets positioned on the front, sides, or back of the garment to target different muscle groups.
Compression features integrated into sleeves, pant legs, or body panels instead of or in addition to pockets.
Garments configured as full-body versions covering the torso, arms, legs, and head to provide extended therapeutic coverage and enhanced thematic or costume effects.
Sensor modules configured for augmented reality (AR) or virtual reality (VR) interactions.
Garments incorporating thermally active textiles to provide warming or cooling effects in conjunction with compression.
Reversible garments offering two distinct themes or colorways.

REFERENCE NUMERALS LIST

| Ref. No. | Part Name | Description |
|---|---|---|
| 10 | Garment | Overall multifunctional garment body |
| 12 | Hood | Head-covering portion, may be fixed or detachable |
| 16 | Fastener | Attachment means for accessories |
| 18 | Flippable Pocket Edge | Edge that can be inverted to open/close |
| | | hand coverage |
| 20 | Pocket | Closed-end pocket for hands/forearms |
| 22 | Sleeve Edge | Opening for hand extension |
| 24 | Diagonal Seam | Seam attaching forearm cradle |
| 26 | Sensor Module | Embedded or detachable sensor unit |
| 28 | Decorative Element/ | Thematic additions such as ears, tails, |
| | Accessory | crowns, wings |
| 30 | Forearm Cradle | Section for forearm compression |
| 32 | Fabric Panel | Folded fabric forming part of cradle |
| 34 | Pocket Seam | Seam defining upper pocket boundary |
| 38 | First Attachment Point | Start of diagonal seam |
| 40 | Second Attachment Point | End of diagonal seam |

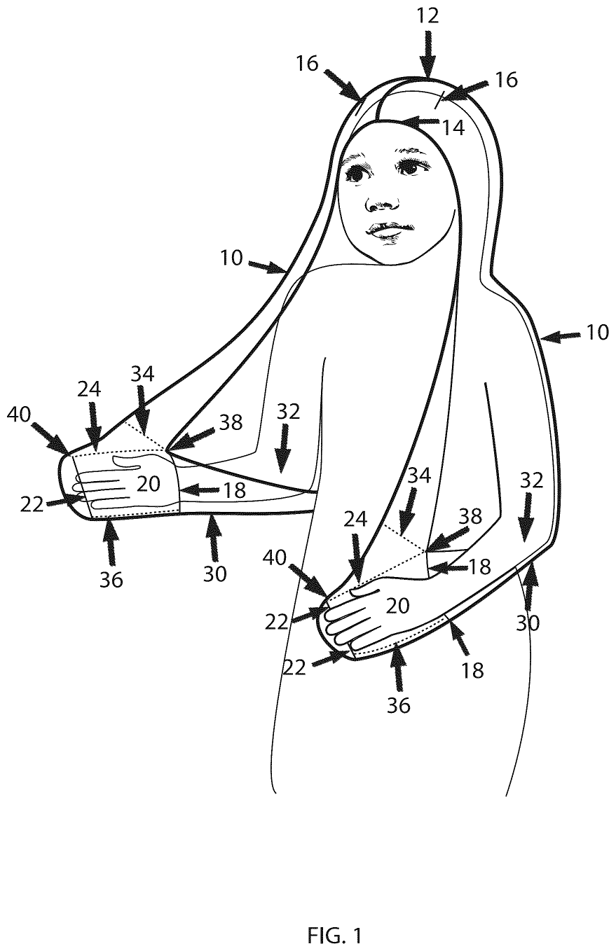

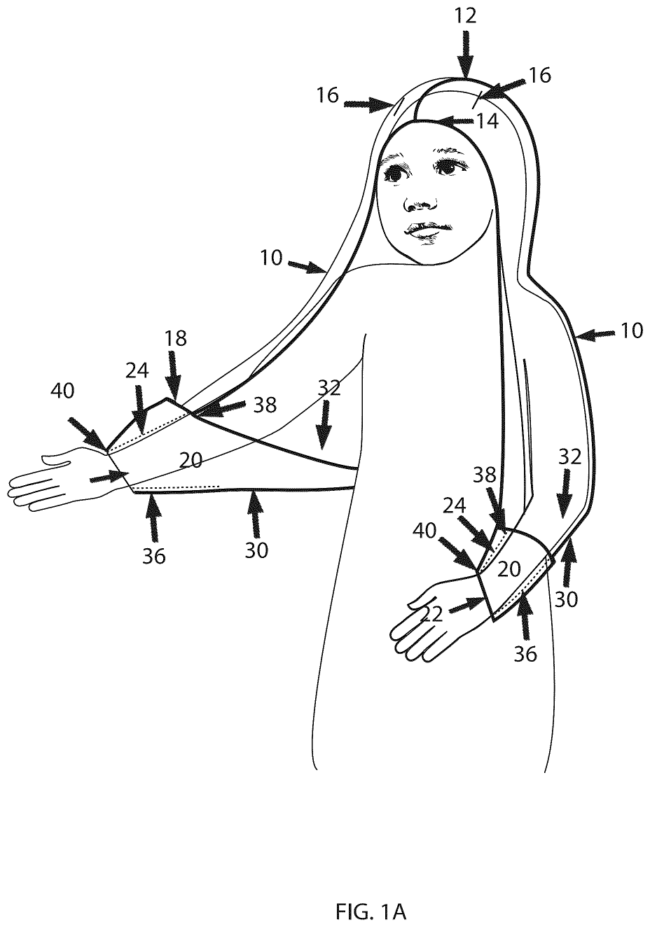

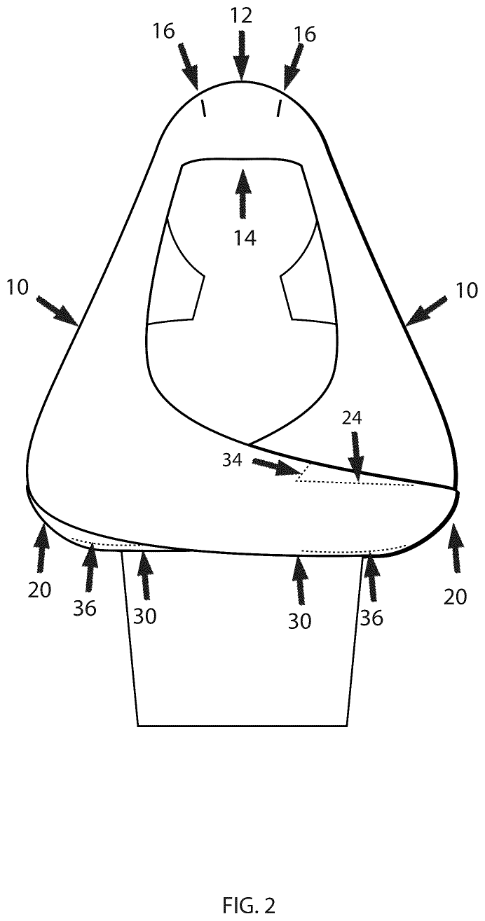

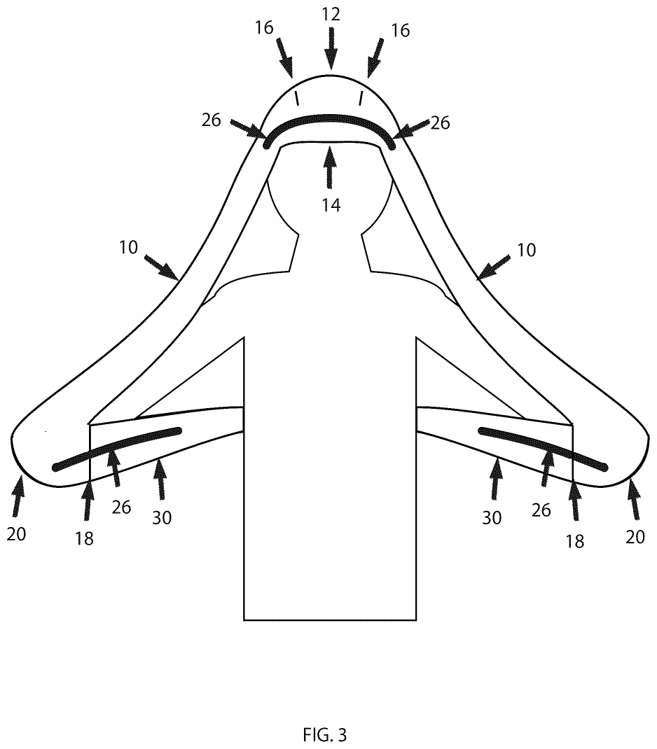

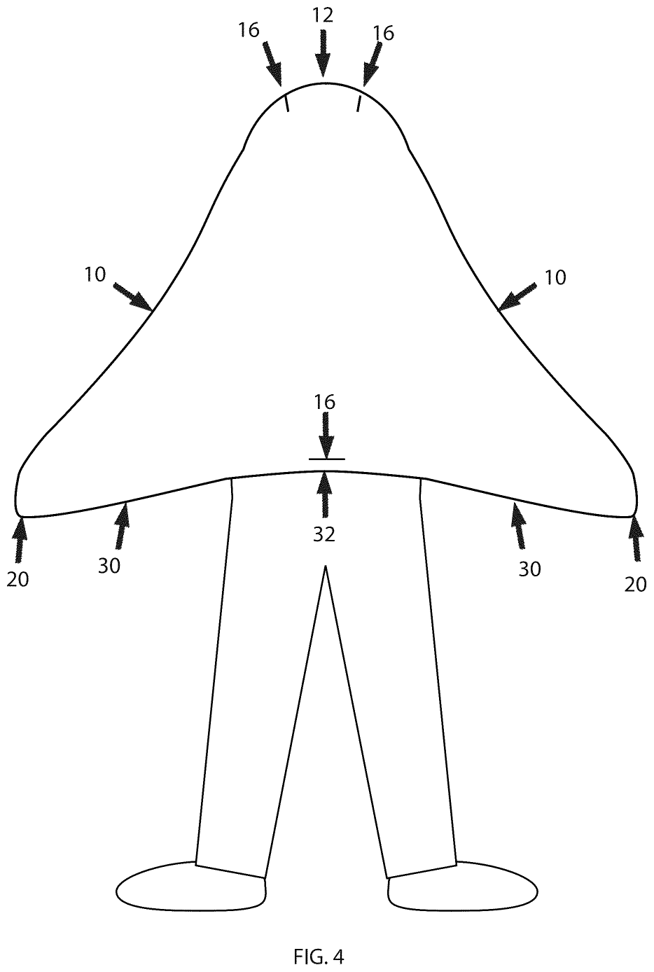

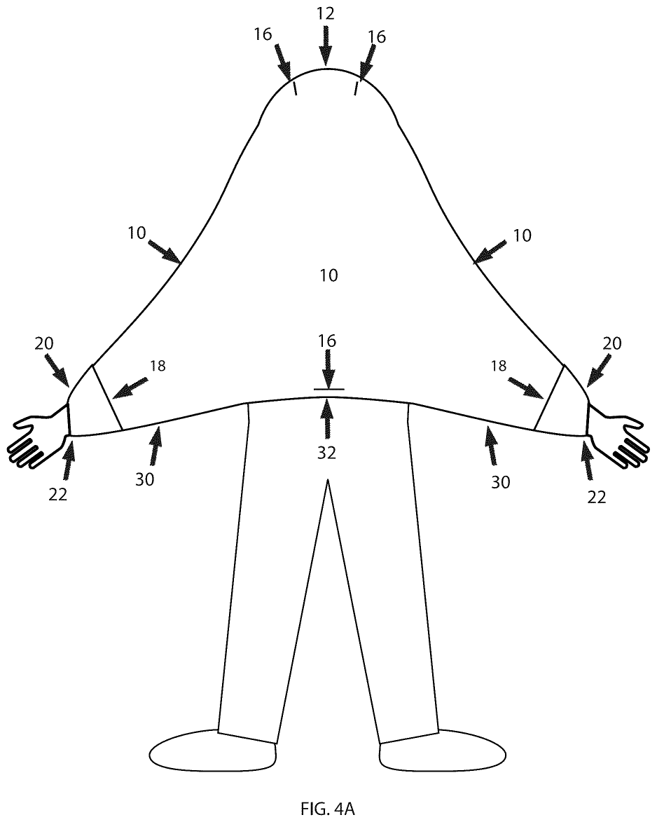

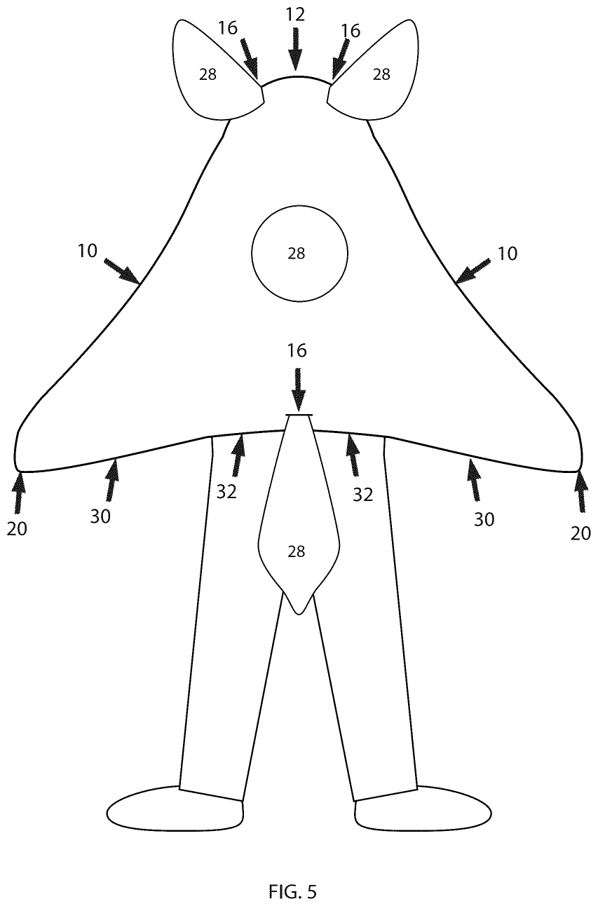

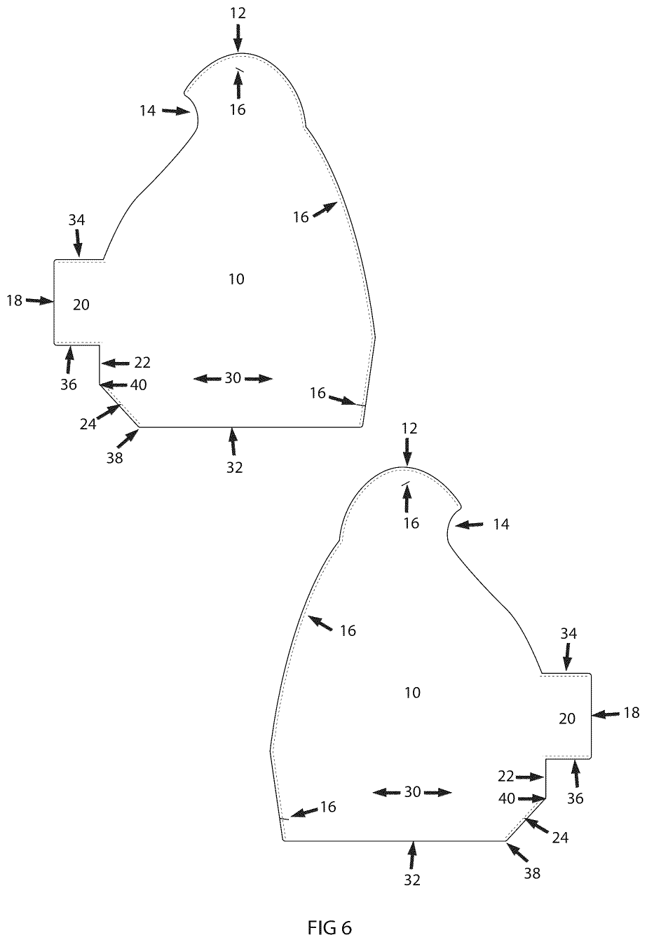

FIGURE-TO-DESCRIPTION MAPPING TABLE

| Figure No. | Title/View |
|---|---|
| FIG. 1 | A front perspective view of one embodiment of the multifunctional garment, |
| | showing the hood, closed-end pockets, and hands covered inside the pockets |
| FIG. 1A | A front perspective view of the garment of FIG. 1, showing the flippable pocket |
| | edges inverted to allow hands to be uncovered |
| FIG. 2 | Front view of the garment with hands and arms applying force to the closed |
| | pockets, arms are crossed in front of the wearer for increased proprioceptive and |
| | tactile sensory experience |
| FIG. 3 | Front view of the garment hands covered in the enclosed pockets, showing the |
| | placement of sensor modules, which may attach to the inside of the garment. |
| | Indicators show where detachable accessories can be applied |
| FIG. 4 | Rear view of the garment with hands covered in the enclosed pockets, exerting |
| | force outwards for increased proprioceptive and tactile sensory experience |
| FIG. 4A | Rear view of the garment with hands uncovered so hands can be used freely. |
| | Indicators show where detachable accessories can be applied |
| FIG. 5 | Rear view of the garment with hands covered in the enclosed pockets, with |
| | attached ears and tail accessories, facilitating imaginative play |
| FIG. 6 | Pattern of garment prior to assembly |

1. An elastic garment comprising a body portion configured to cover a wearer's torso and head; a pair of closed-end pockets positioned to receive the wearer's hands and arms, each pocket comprising a pocket edge configured to be flip-able from the inside to the outside of the pocket, thereby allowing the wearer to uncover or cover their hands at will; wherein the elastic construction is configured such that when the wearer pushes against the interior of the pockets, tension in the fabric increases pressure across the head, arms, and torso; and wherein the garment is further configurable to incorporate various character designs and thematic elements through graphic surface embellishments, including printing, embroidery, or sewn additions, to support imaginative play, role-play activities, and customization.

2. The garment of claim 1, further comprising one or more detachable imaginative play accessories selected from the group consisting of ears, tails, crowns, wings, and other character-themed elements.

3. The garment of claim 1 or 2, wherein the elastic fabric comprises a knit or woven textile with stretch properties sufficient to provide varying levels of compression based on user-applied force.

4. The garment of any preceding claim, wherein the closed-end pockets are positioned near sleeve termini or along the upper torso such that pushing against the pockets directs compressive forces to the wearer's shoulders, arms, and upper torso.

5. The garment of any preceding claim, wherein the hood is constructed from the same elastic fabric as the body portion and configured to provide proprioceptive and deep pressure input around the wearer's head when the wearer exerts force on the pockets.

6. The garment of any preceding claim, wherein the one or more detachable imaginative play accessories are removably attached via hook-and-loop fasteners, snaps, zippers, or other mechanical fasteners.

7. The garment of any preceding claim, further comprising embedded or attachable sensor modules configured to monitor biometric data or wearer motion.

8. The garment of claim 7, wherein the sensor modules are configured to communicate wirelessly with an external device to transmit biometric or motion data.

9. The garment of any preceding claim, wherein the garment is configured to interface with a digital application that provides visual, auditory, or haptic feedback based on biometric, motion, or location inputs from the wearer.

10. The garment of any preceding claim, wherein the garment serves as a therapeutic tool for improving proprioception, body awareness, emotional regulation, or motor coordination.

11. The garment of any preceding claim, wherein the garment serves as a recreational costume enabling imaginative role-play and creative expression through its customizable accessory features and graphic embellishments.

12. The garment of any preceding claim, wherein the wearer controls the degree of compression by adjusting the force applied to the closed-end pockets, allowing self-directed sensory input without the use of straps, buckles, inflatable elements, or weights.

13. The garment of any preceding claim, wherein the garment's materials and design allow for repeated stretching and recovery without loss of elasticity or structural integrity.

14. The garment of any preceding claim, wherein each pocket comprises a pocket edge that can be flipped from the inside to the outside, allowing the wearer to uncover or cover their hands at will, offering additional control over the garment's function.

15. The garment of any preceding claim, wherein the graphic surface embellishments, including printed images, embroidered designs, or sewn-on patches, can be applied to create a variety of character themes, allowing for personalization and potential licensing opportunities for customized designs and interactive play.
